(19) United States
(12) Patent Application Publication
(10) Pub. No.: US 2013/0304116 A1
(43) Pub. Date: Nov. 14, 2013

(54) PLUG

(71) Applicant: FUJIFILM CORPORATION, Tokyo (JP)

(72) Inventor: Kenji YAMANE, Ashigarakami-gun (JP)

(73) Assignee: FUJIFILM Corporation, Tokyo (JP)

(21) Appl. No.: 13/874,501

(22) Filed: May 1, 2013

(30) Foreign Application Priority Data
May 9, 2012 (JP) 2012-107508

(51) Int. Cl. A61B 1/00

(52) U.S. Cl. CPC A61B 1/00137
USPC 606/205

(57) ABSTRACT

The fixed valve and the attachable and detachable valve are connected to each other via a connecting band, and both are integrally made of elastomer. A frame body is constituted of a rigid body made of plastic. A flange and a receiving portion are formed on an upper portion of the frame body. A second flange is formed on a lower end of the fixed valve. The fixed valve is installed on the frame body by engaging the second flange with flange. The second flange is prevented from being separated from the flange of the second flange by interposing a part of the second flange between the flange and the receiving portion. The fixed valve is securely installed on the frame body, whereby the fixed valve is prevented from uncoupling when removing a treatment tool.

### BACKGROUND OF THE INVENTION

1. Field of the Invention

The present invention relates to a plug mounted on a channel opening portion of a treatment tool channel of an endoscope.

2. Description of the Related Art

In the field of medicine, various treatments aside from the intracorporeal observation have been executed with respect to a diseased part by inserting an insertion portion of an endoscope into the body of a patient. Specifically, various treatments such as excising or sampling of a diseased part are carried out by inserting various treatment tools such as a forceps or an incision tool from a channel opening portion provided in an operation portion of the endoscope into a treatment tool channel inside an insertion portion and protruding it from an insertion portion tip end.

A forceps plug (plug) enabling the treatment tool to be inserted when performing treatments is mounted on the channel opening portion (see JP2007-185371A, JP2005-177080A and JP2005-224529A). This kind of forceps plug prevents body fluid, sordes, saline for washing the inside of the body, or the like in vivo from flowing backward through the treatment tool channel and leaking outside through the channel opening portion due to the inner pressure changes in vivo. As such a forceps plug, a forceps plug having a frame body provided with a lower valve/upper valve double-structure valve element has been disclosed in JP2007-185371A, JP2005-177080A or JP2005-224529A. For example, in the forceps plug disclosed in JP2007-185371A or JP2005-177080A, the lower valve includes a valve film which is provided with a treatment tool insertion hole having a large diameter and used in the case of using a treatment tool having a large diameter. In addition, the upper valve includes a valve film which is provided with a slit and used in the case of inserting a treatment tool having a diameter small enough to cause a gap between the treatment tool and the treatment tool insertion hole of the lower valve. Also, when the treatment tool having a large diameter is used, the upper valve is removed from the frame body. Thereby, generation of insertion resistance due to the upper valve is restrained, and therefore, operability thereof is improved. Upon comparison with the forceps plug disclosed in JP2005-177080A, the forceps plug disclosed in JP2005-224529A is different in that the positional relationship between the treatment tool insertion hole and the slit in the lower valve and the upper valve is reversed.

### SUMMARY OF THE INVENTION

In the forceps plug disclosed in JP2007-185371A, the upper valve and the lower valve are integrally made of a rubber member, and therefore, the configuration thereof is simple. However, since both valves are made of a rubber member, joint strength between the upper valve and the lower valve is weak. Therefore, there is possibility that the lower valve may be uncoupled from the channel opening portion together with the treatment tool, or the upper valve may be uncoupled from the lower valve by frictional resistance or hooking when removing the treatment tool. If the engagement between the lower valve and the upper valve is released and uncoupled, there is a risk of infection due to dispersed body fluid or the like.

Furthermore, the forceps plug disclosed in JP2005-177080A or JP2005-224529A is a disposable type. Therefore, the frame body is provided with a breaking portion, and reuse of the forceps plug is prevented by breaking the breaking portion after use so as to restrict the remount of the channel opening portion. Thus, since the upper valve is engaged with the frame body having rigidity, it is possible to maintain joint strength therebetween compared to the engagement between parts made of a rubber member as described in JP2007-185371A. Therefore, the upper valve is prevented from uncoupling together with the treatment tool when removing the treatment tool.

However, the forceps plug disclosed in JP2005-177080A or JP2005-224529A has a structure where the upper valve is engaged with an upper portion of the frame body. Therefore, when the treatment tool is inserted in a state of removing the upper valve, there is a possibility that the upper portion of the frame body may be in contact with the treatment tool and body fluid or the like stuck to the treatment tool may be in contact with the upper portion of the frame body. Therefore, it is necessary for the frame body to be made of a material having compatibility with the living body, whereby there is a problem in that available material for the frame body is limited. In addition, it is necessary for the frame body to be provided with an engaging portion or an engaging protrusion for the valves on its inner circumferential surface in order to prevent the lower valve or the upper valve from uncoupling from an inner portion of the frame body. Subsequently, a plurality of undercut parts are generated at molds when molding the frame body, and therefore, manufacturing cost for the molds is increased. Also, there is a problem in that molding suitability is deteriorated due to the plurality of undercut parts. The undercut part means a portion which requires partially moving the molds when separating molded products from the molds.

Moreover, the forceps plug disclosed in JP2005-177080A or JP2005-224529A is constituted of three parts of the upper valve, the lower valve and the frame body, and therefore, it is necessary to insert the lower valve into the frame body, to mount the upper valve on the frame body or to engage an extending portion of the upper valve with the frame body. As a result, there is a problem in that man-hours for assembly are increased.

The present invention has been made in view of the above-mentioned problems and an object of the present invention is to provide a plug which is easy to assemble, capable of forming a frame body with an arbitrary material, and also, capable of preventing an upper valve and a lower valve from uncoupling together with a treatment tool when removing the treatment tool.

In order to achieve the object described above, there is provided a plug that is mounted on an channel opening portion of a treatment tool channel inside an endoscope and allows an insertion of a treatment tool in the channel opening portion and has a fixed valve, an attachable and detachable valve and a frame body. The fixed valve is made of elastomer having a cylindrical shape and includes a valve film having an insertion hole for the treatment tool, an engaging portion on an outer circumferential surface and an attachable and detachable valve mounting flange on an upper portion. Also, the fixed valve is provided in the channel opening portion such that a lower end thereof is in contact with the channel opening portion.

The attachable and detachable valve is made of elastomer having a cap shape and includes a valve film having a slit allowing the insertion of the treatment tool and an annular protrusion on an inner circumferential surface to be engaged with the attachable and detachable valve mounting flange. Also, the attachable and detachable valve is attachably and detachably connected to an upper portion of the fixed valve by engaging the annular protrusion with the attachable and detachable valve mounting flange.

The frame body is made of plastic and includes an engaging claw to be engaged with the channel opening portion in a state where a lower end of the fixed valve inserted is in contact with the channel opening portion and a fixed valve mounting flange to be engaged with the engaging portion of the fixed valve. Also, the frame body holds the fixed valve by engaging the engaging portion with the fixed valve mounting flange.

In this application, elastomer means a material which is a synthetic resin and has a property of returning to an original shape even in the case of being largely bent. Rubber is an example of the elastomer. In addition, plastic means a material which is a synthetic resin and has a property of plastic deformation or breaking in the case of being largely bent, despite having rigidity. Thermoplastic resin is an example of the plastic.

It is preferable that the plug is provided with a connecting portion made of elastomer which is integrally formed with the fixed valve and the attachable and detachable valve so as to combine the fixed valve with the attachable and detachable valve. In this case, since the fixed valve, attachable and detachable valve and connecting portion are integrally formed, it is easy to manufacture, and also, it is simple to mount it on the frame body.

It is preferable that the plug includes a knob portion that is formed on an outer circumferential surface of the attachable and detachable valve and extends in a diametrical direction of the attachable and detachable valve on an opposite side to the connecting portion, an engaging claw that is formed on the outer circumferential surface of the frame body on an opposite side of the connecting portion in a state where the fixed valve is mounted on the frame body, and extends in a diametrical direction of the frame body as well as protruding in an opposite direction to a pulling-out direction of the treatment tool, and an engaging hole that is formed on the knob portion at a position where the engaging claw is to be engaged in a state where the knob portion is set to be along the outer circumferential surface of the frame body, in which, in a state where the engaging hole of the knob portion is engaged with the engaging claw, a portion corresponding to the length from the attachable and detachable valve of the knob portion to the engaging hole constitutes the connecting portion. In this case, since the attachable and detachable valve is securely connected to the frame body by engaging the engaging claw with the engaging hole of the knob portion, the attachable and detachable valve is prevented from uncoupling from the fixed valve together with the treatment tool when removing the treatment tool.

It is preferable that the plug includes a pair of engaging claws that are formed on the outer circumferential surface of the frame body and extend in a diametrical direction of the frame body as well as protruding in an opposite direction to a pulling-out direction of the treatment tool, a pair of knob portions that are formed on an outer circumferential surface of the attachable and detachable valve and extend in a diametrical direction of the attachable and detachable valve, and engaging holes that are formed on the knob portions at positions where the engaging claws are to be engaged in a state where the knob portion is set to be along the outer circumferential surface of the frame body. In this case, since the attachable and detachable valve is fixed to the frame body by the knob portion, it is possible to securely mount the attachable and detachable valve on the frame body. Therefore, the attachable and detachable valve is prevented from uncoupling together with the treatment tool when removing the treatment tool.

It is preferable that the plug includes a slip-out prevention receiving portion that is integrally formed with the frame body on an outer circumferential surface of the frame body at a lower side of the fixed valve mounting flange of the frame body, and partially covers an outer circumferential surface of the engaging portion. In this case, the engaging portion of the fixed valve is firmly held by the frame body due to the slip-out prevention receiving portion, and therefore, the fixed valve is prevented from uncoupling from the frame body.

It is preferable that the frame body is unable to be separated from the fixed valve by bonding, welding or swaging of partially deforming the frame body. In this case, the frame body is unable to be separated from the fixed valve, and therefore, reuse of the fixed valve or attachable and detachable valve is prevented.

It is preferable that the frame body includes knob portions to be separated that protrude in a diametrical direction from an outer circumferential surface on the channel opening portion side, and are connected to the frame body via connection parts positioned at both sides in a circumferential direction, a notch that is formed on at least one of the connection parts in a direction of a cylindrical center of the frame body, and a fragile portion that is constituted of the connection part notched, and used for separating the knob portion to be separated. In this case, since the knob portion to be separated is separated from the frame body by using the fragile portion, a lower portion of the frame body is opened widely. Therefore, it is not possible to remount the frame body on the channel opening portion, whereby reuse of the frame body is prevented.

It is preferable that the frame body has a side opening that is in contact with the fragile portion and formed in the frame body. In this case, when the knob portion to be separated is separated from the frame body, the wide opening including the side opening is formed at the lower portion of the frame body. Therefore, it is possible to simply remove the frame body from the channel opening portion. In addition, when the frame body is remounted, the wide opening including the side opening is formed on the frame body. Therefore, reuse of the frame body is reliably prevented.

It is preferable that two fixed claws formed at intervals of approximately 180 degrees in the circumferential direction of the frame body constitute the engaging claw portions, and the fixed claws are formed at positions symmetric with respect to a center line of the knob portion to be separated in the diametrical direction. In this case, when the knob portion to be separated is separated from the frame body, the engagement between the two fixed claws and the channel opening portion is easily released. Therefore, the frame body is simply removed from the channel opening portion.

According to the plug of the invention, it is possible for a frame body to be made of an arbitrary material, and also, to be securely fixed to a channel opening portion. In addition, there is no possibility that an attachable and detachable valve or a fixed valve may be uncoupled together with a treatment tool by frictional resistance or hooking between the attachable and detachable valve or the fixed valve and the treatment tool when removing the treatment tool.

### BRIEF DESCRIPTION OF THE DRAWINGS

FIG. 1 is a perspective view which shows a schematic configuration of an endoscope system and partially includes a front view.

FIG. 2 is a perspective view showing a plug in an exploded state.

FIG. 3 is a perspective view showing the whole plug.

FIG. 4 is a cross-sectional view of the plug.

FIG. 5 is a side view of a frame body which shows a bending limit of a knob portion to be separated when bent downwardly.

FIG. 6 is a side view of a frame body which shows a broken state of the knob portion to be separated when bent upwardly.

FIG. 7 is a perspective view showing a plug according to a second embodiment in an exploded state.

FIG. 8 is a cross-sectional view of the plug according to the second embodiment.

FIG. 9 is a perspective view showing a plug in an exploded state where a frame body is a part of a conduit.

FIG. 10 is a perspective view showing the whole plug.

FIG. 11 is a cross-sectional view of the plug.

FIG. 12 is a cross-sectional view showing a state of pulling out a treatment tool from the plug.

### DESCRIPTION OF THE PREFERRED EMBODIMENTS

### First Embodiment

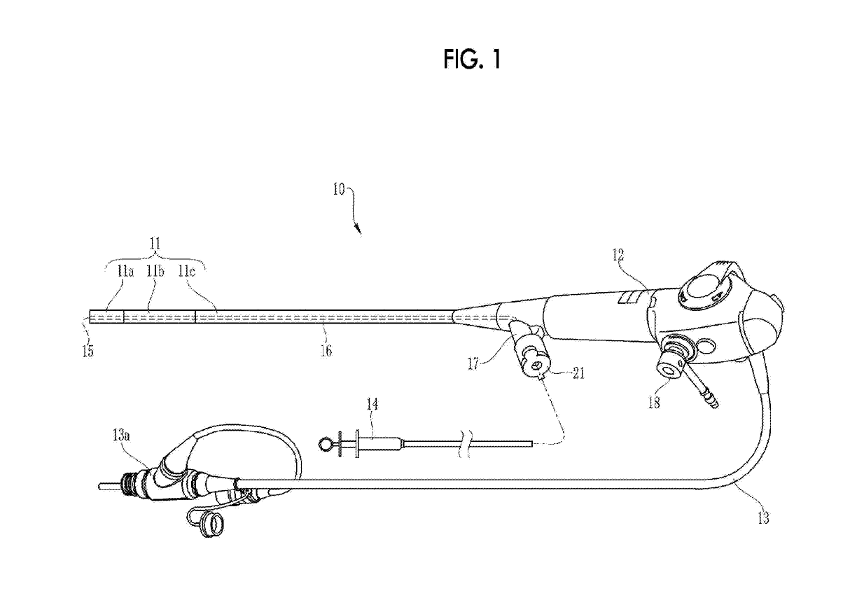

As shown in FIG. 1, an endoscope 10 is a bronchoscope inserted into a trachea, for example, and includes an insertion portion 11 inserted into a trachea, an operation portion 12 continuously provided at a base end portion of the insertion portion 11 and an universal code 13 joined to the operation portion 12. The universal code 13 is joined to a processor unit, a light source device (not shown) or the like via a complex type connector 13a.

The insertion portion 11 is divided into a hard distal end portion 11a, a bending portion 11b freely bendable and a flexible tube portion 11c having flexibility in order from a tip end side to the base end side. An observation window or an illumination window (not shown) aside from a treatment tool outlet 15 which is used as an outlet of a treatment tool 14 such as a forceps are provided on a tip end surface of the hard distal end portion 11a. An image sensor (not shown) or the like is disposed inside the observation window, and an optical fiber cable (not shown) is disposed inside the illumination window. A signal wire of the image sensor and the optical fiber cable pass through the inside of the insertion portion 11, the universal code 13 and the connector 13a, and are respectively joined to the processor unit and the light source device.

A treatment tool channel 16 to insert the treatment tool 14 is provided inside the insertion portion 11. One end of the treatment tool channel 16 is joined to the treatment tool outlet 15 and the other end is joined to a channel opening portion 17 of the operation portion 12. The treatment tool channel 16 is used as a passage to suck body fluid such as blood or solid matter such as internal sordes through the treatment tool outlet 15 as well. A suction channel (not shown) branched from the treatment tool channel 16 is provided in the operation portion 12, and the suction passage is joined to a suction button 18 of the operation portion 12.

The suction button 18 is joined to a negative pressure source (not shown) in an outside of the operation portion 12. The suction button 18 switches communication and cutoff states between the suction passage and the negative pressure source corresponding to a pressing operation or a release of the pressing operation.

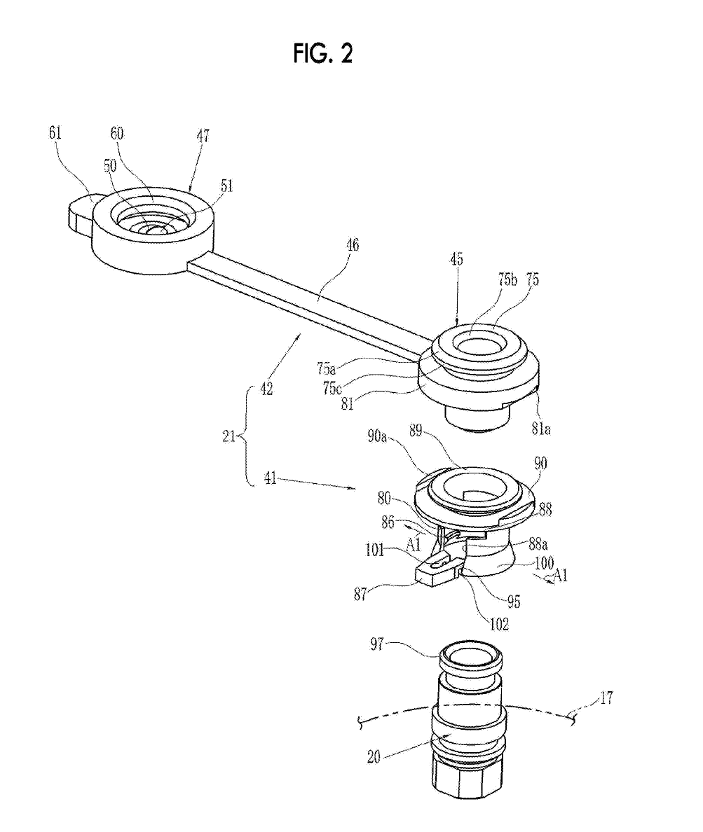

A disposable plug (forceps plug) 21 through which the treatment tool 14 (see FIG. 1) is inserted is mounted on the channel opening portion 17. As shown in FIG. 2, the channel opening portion 17 has a mouthpiece 20. The plug 21 is mounted on the channel opening portion 17 via the mouthpiece 20.

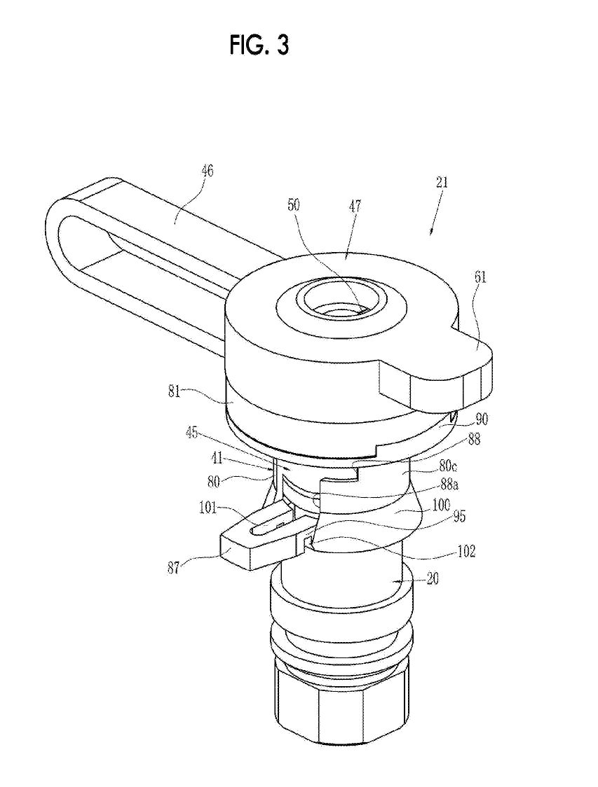

As shown in FIGS. 2 and 3, when performing a treatment using the treatment tool 14 (see FIG. 1), the plug 21 prevents body fluid or the like in vivo from flowing backward through through the treatment tool channel 16 and leaking outside through the mouthpiece 20. The plug 21 has a frame body 41 and a valve element 42 held by the frame body 41. The valve element 42 is provided in the mouthpiece 20 and allows an insertion of the treatment tool 14 when inserting the treatment tool 14.

The valve element 42 has a fixed valve 45, a connecting band (connecting portion) 46, and an attachable and detachable valve 47. The connecting band 46 connects the fixed valve 45 to the attachable and detachable valve 47. The valve element 42 is made of elastomer such as rubber. The attachable and detachable valve 47 is provided with a valve film 51 having a slit 50 and is formed in a cap shape. An annular protrusion 60 protruding inside is formed on an inner circumferential surface of the attachable and detachable valve 47. In addition, a knob portion for removing 61 is integrally formed with the attachable and detachable valve 47 on an opposite side to the connection side of the connecting band 46 of the attachable and detachable valve 47.

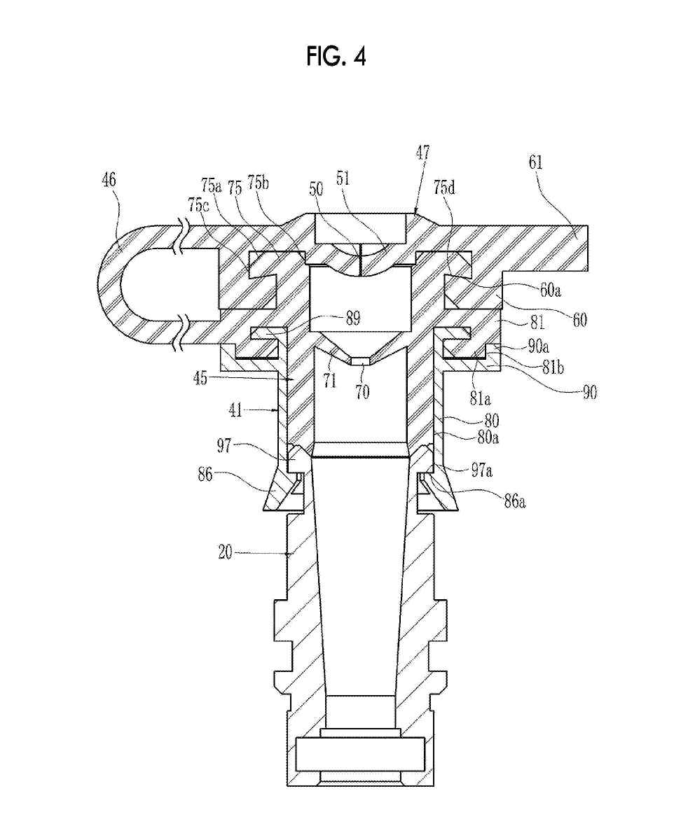

As shown in FIG. 4, the fixed valve 45 is provided with a valve film 71 having a treatment tool insertion hole 70 and is formed in a cylindrical shape. The valve film 71 is recessed in a hemispherical shape or a conical surface shape, and has the treatment tool insertion hole 70 at a central portion thereof As shown in FIG. 2, a first flange (attachable and detachable valve mounting flange) 75 is provided in an upper end of the fixed valve 45. A lower portion of the fixed valve 45 is inserted in a frame main body 80 and is held by an inner circumferential surface 80a thereof The first flange 75 is not inserted in the frame main body 80. Also, a second flange 81 is provided in an outer circumferential surface of the fixed valve 45. The second flange 81 has an engaging portion 81a to be engaged with and fixed to the frame body 41.

The first flange 75 has a flange outer-side inclined surface 75a on the outer circumferential surface side thereof In addition, the first flange 75 has a flange inner-side inclined surface 75b on an inner circumferential surface side thereof When the first flange 75 is covered with the attachable and detachable valve 47, the annular protrusion 60 of the attachable and detachable valve 47 is extendedly deformed so as to get over the flange outer-side inclined surface 75a and the outer circumferential surface 75c, and also, an engaging surface 60a of the annular protrusion 60 is engaged with an engaging surface 75d of the first flange 75. The engaging surface 75d and the engaging surface 60a are inclined obliquely downward toward the outside.

As shown in FIG. 2, the frame body 41 has a frame main body 80 having an approximately cylindrical shape, a fixed claw 86 formed in the frame main body 80, a knob portion to be separated 87, a side opening 88, a flange 89 and a receiving portion 90, and is integrally molded with plastic. The fixed claw 86 is formed on an inner circumferential surface of a lower end portion of the framed main body 80. Also, on an outer circumferential surface of the frame main body 80, the knob portion to be separated 87 and the side opening 88 are formed in order from the lower end. The flange 89 as a fixed valve mounting portion is formed on an upper end surface of the frame main body 80.

As shown in FIG. 4, in the frame body 41, the fixed valve 45 is mounted on an opening portion of the mouthpiece 20 in a state where the fixed valve 45 is in contact with an opening of the mouthpiece 20. The receiving portion 90 partially covering an outer circumferential surface of the engaging portion 81a is provided on the outer circumferential surface of the frame body 41 so as to enhance locking strength between the engaging portion 81a and the flange 89 and to make the fixed valve 45 be not separated from the frame body 41. The receiving portion 90 is integrally formed with the frame body 41. The receiving portion 90 has a convex portion 90a fit into a concave portion 81b of the second flange 81. Here, it is preferable that the engaging portion 81a and the receiving portion 90 are welded or bonded to each other. In this case, the engaging portion 81a is unable to be separated from the receiving portion 90, and therefore, reuse of the valve element 42 is prevented.

As shown in FIGS. 3 and 4, the side opening 88 has a joining opening 88a at the lower portion thereof. The joining opening 88a extends to a fragile portion 95 described below. Two fixed claws 86 are formed on the frame main body 80 at a pitch of 180 degrees in a circumferential direction. The fixed claws 86 get over a flange 97 of the mouthpiece 20 when the frame main body 80 is mounted on the mouthpiece 20. Thereby, an engaging surface 86a of the fixed claw 86 is engaged with a flange engaging surface 97a. Furthermore, in this case, the pitch of 180 degrees is an angle including manufacturing error (±5 degrees) caused at the time of forming the fixed claw 86.

An outer circumferential surface 80c of a lower end portion of the frame main body 80 is an tapered surface 100 which is gradually widened toward the lower end. By providing the tapered surface 100, the thickness of the lower end portion of the frame main body 80 is secured, and also, the strength of the lower end portion of the frame main body 80 is maintained. The knob portion to be separated 87 protruding in a radial direction of the frame main body 80 is formed on the tapered surface 100. The two fixed claws 86 are formed at positions symmetric with respect to a center line of the knob portion to be separated 87 in the radial direction. In a horizontal plane including the two fixed claws 86, the knob portion to be separated 87 is formed in a U shape having a widenable opening 101.

As shown in FIG. 3, in a direction of a cylindrical center of the frame main body 80, notches 102 are respectively formed at parts which are near both sides of joining portions between the knob portion to be separated 87 and the frame main body 80 and is located on an outer side in a radial direction from the outer circumferential surface 80c of the frame main body 80, from a lower end toward an upper side. In the direction of the cylindrical center of the frame main body 80, connection parts between the knob portions to be separated 87 and the frame main body 80 are formed shortly due to the notches 102. The connection parts formed shortly are the fragile portions 95 to separate the knob portion to be separated 87 from the frame main body 80.

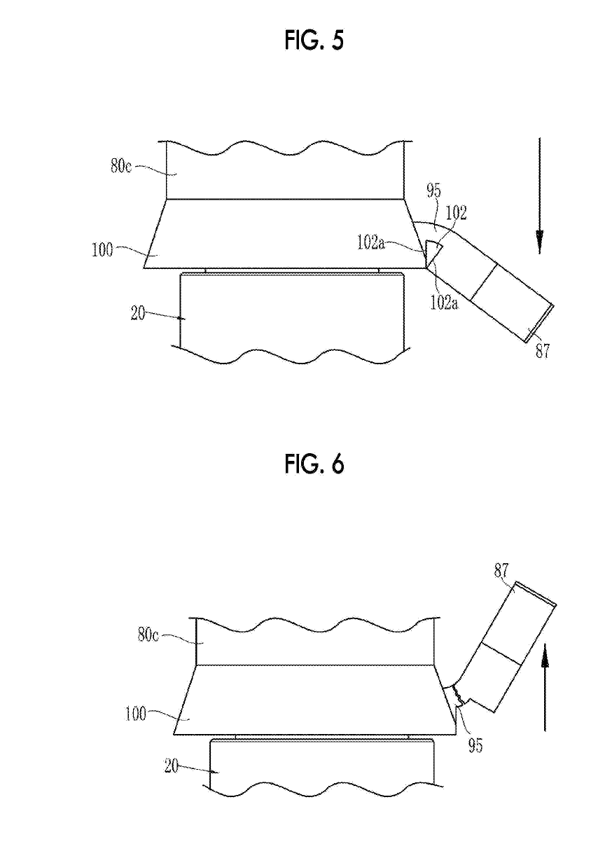

As shown in FIG. 5, in a pair of notch surfaces 102a facing each other, when the knob portion to be separated 87 is pushed downward, the notch surfaces 102a are in contact with each other, and therefore, functioning as a stopper to regulate further bending. On the other hand, when the knob portion to be separated 87 is pushed upwardly, the notch surfaces 102a are not in contact with each other, and therefore, it is possible to largely bend the knob portion to be separated 87 upwardly, as shown in FIG. 6. Due to the bending deformation, the fragile portions 95 are broken after plastic deformation. In addition, the notches 102 may be formed on both sides in the direction of the cylindrical center, instead of forming it on one side in the direction thereof.

At the time of using the endoscope 10, the plug 21 is mounted on the mouthpiece 20 of the operation portion 12 of the endoscope 10, as shown in FIG. 1. This mounting process is carried out by pushing the lower end portion of the plug 21 so as to be externally fit to the flange 97 of the mouthpiece 20, as shown in FIG. 2. Thereby, the lower end portion of the frame main body 80 is deformed such that the widenable opening 101 of the knob portion to be separated 87 is opened, and therefore, it is possible for the two fixed claws 86 to move in a direction of an arrow A1 and to easily get over the flange 97. In the case where the fixed claw 86 gets over the flange 97, the engaging surfaces 86a and 97a (see FIG. 4) of the fixed claw 86 and the flange 97 abut on each other and are reliably engaged. Therefore, it is not possible to remove the frame main body 80 from the mouthpiece 20 in a state of being engaged with each other.

In a state where the attachable and detachable valve 47 is fit to the flange 75 of the fixed valve 45 and the treatment tool 14 (see FIG. 1) is not used, the slits 50 of the attachable and detachable valve 47 are in close contact with each other by a resilient force of the attachable and detachable valve 47, and therefore, air- and water-tightness thereof are secured.

When one of the treatment tools 14 having a relatively small diameter is used, it is possible to insert the treatment tool 14 through the slit 50 of the attachable and detachable valve 47 in a state of fitting the attachable and detachable valve 47 into the upper portion of the fixed valve 45. When the treatment tool 14 is inserted through the slit 50, surface of the slit 50 is in close contact with the outer circumferential surface of the treatment tool 14 by a resilient force of the attachable and detachable valve 47. Therefore, even if the insertion portion 11 is inserted into the body of a patient, blood, body fluid or the like is prevented from leaking outside through the plug 21. Furthermore, When the treatment tool 14 having a large diameter is used, it is also possible to insert the treatment tool 14 through the treatment tool insertion hole 70 of the fixed valve 45 by removing the attachable and detachable valve 47 from the fixed valve 45.

As shown in FIG. 4, not the attachable and detachable valve 47 but only the fixed valve 45 is mounted on the frame body 41, and the attachable and detachable valve 47 is directly attached to/detached from the upper portion of the fixed valve 45. Therefore, when the treatment tool 14 (see FIG. 1) is inserted, the entire part of the conduit inside the plug 21 where the treatment tool 14 is inserted is made of elastomer regardless of removing the attachable and detachable valve 47 from the fixed valve 45. Thereby, it is possible that the frame body 41 is not in contact with the treatment tool 14, and a material is not limited to one having compatibility with the living body. Also, it is possible to select a material allowing a more secure engagement.

Since a part of the outer circumferential surface of the engaging portion 81a is covered by the receiving portion 90 so as to enhance the locking strength between the engaging portion 81a and the flange 89 and prevent the fixed valve 45 from being separated from the frame body 41, the fixed valve 45 is prevented from uncoupling together with the treatment tool 14 when removing the treatment tool 14. In addition, the engaging surface 75d and the engaging surface 60a are inclined obliquely downward toward the outside, whereby the engagement therebetween is maintained in a direction where the annular protrusion 60 bites into. Therefore, the attachable and detachable valve 47 is prevented from uncoupling together with the treatment tool 14.

The plug 21 is uncoupled from the mouthpiece 20 after the endoscope 10 is used, and then the endoscope 10 is cleaned. When the plug 21 is removed from the mouthpiece 20, by gripping the knob portion to be separated 87 and lifting it upward, as shown in FIG. 6, the knob portion to be separated 87 is bended upwardly about the fragile portion 95 and then being broken after plastic deformation.

Since the knob portion to be separated 87 broken is separated from the frame main body 80, the side opening 88 having the joining opening 88a is widely opened to a lower end side. Thereby, it is possible to simply release the engagement between the fixed claw 86 and the flange 97 of the mouthpiece 20, and therefore, it is possible to easily remove the plug 21 from the mouthpiece 20. Also, the joining opening 88a and the side opening 88 are opened in a state where the knob portion to be separated 87 is separated from the frame main body 80. Therefore, it is not possible to mount the plug 21 to the mouthpiece 20 again. Also, even if being mounted again, the plug 21 is easily uncoupled, whereby reuse thereof is prevented.

### Second Embodiment

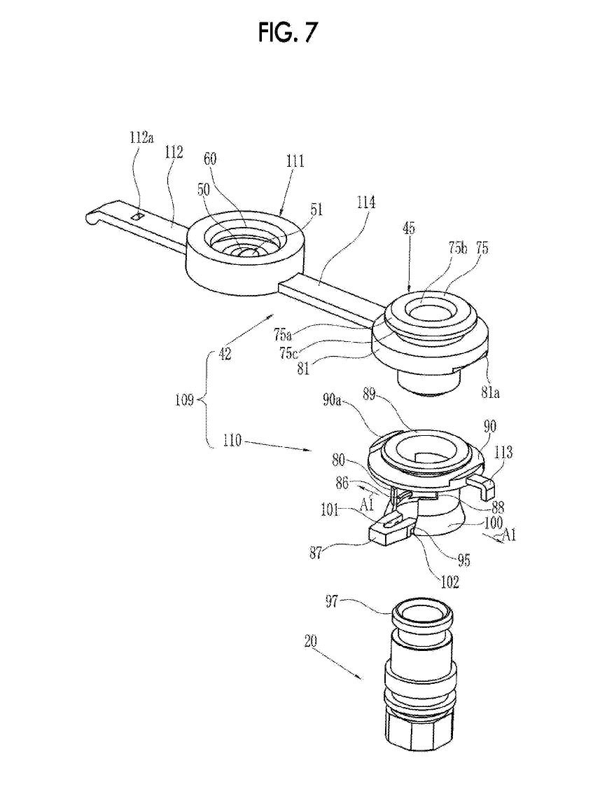

Next, another embodiment where figures of the knob portion of the attachable and detachable valve and the receiving portion of the frame body are changed will be described. As shown in FIG. 7, a knob portion 112 extending in a diametrical direction of the attachable and detachable valve 111 on an opposite side to the connection side of the connecting band 114 is integrally formed with an attachable and detachable valve 111 of the plug 109 of a second embodiment. On the outer circumferential surface of the receiving portion 90 of the frame body 110, an engaging claw 113 which is inserted into an engaging hole 112a of the knob portion 112 and engaged with the knob portion 112 is integrally formed with the receiving portion 90. The engaging claw 113 is formed in a direction different from a direction where the treatment tool 14 (see FIG. 1) is pulled out. In addition, the length of the connecting band 114 is shorter than that of the connecting band 46 in the first embodiment and is the same with the length of the knob portion 112. Furthermore, in the attachable and detachable valve 111 and a frame body 110 of the second embodiment, the entire configuration thereof is the same in the first embodiment except the knob portion 112 and the engaging claw 113. Therefore, the same reference numerals are given to the components having the same configuration, and redundant description thereof is omitted.

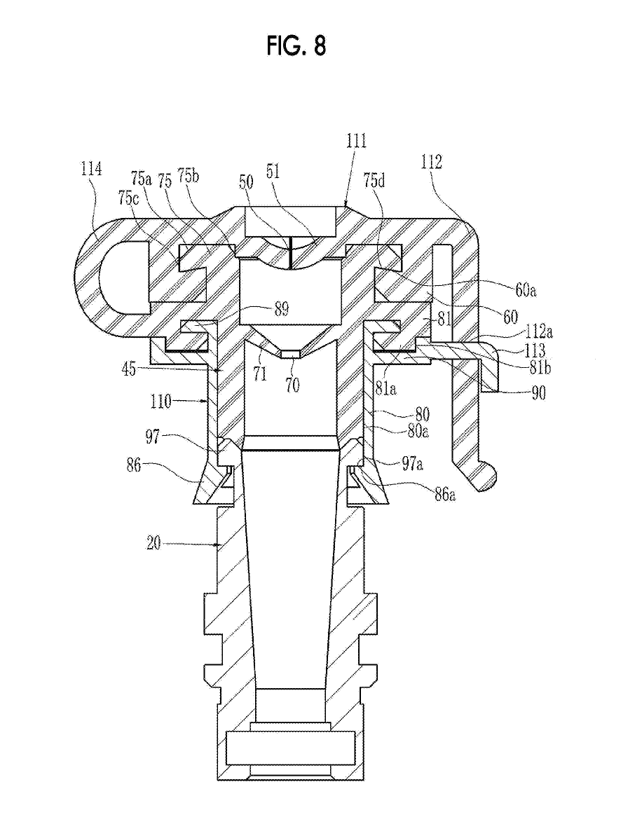

According to the second embodiment, since the knob portion 112 is engaged with the engaging claw 113, as shown in FIG. 8, and therefore, it is possible to securely mount the attachable and detachable valve 111 and the fixed valve 45 on a frame body 109. Thereby, the attachable and detachable valve 111 or the fixed valve 45 is prevented from uncoupling together with the treatment tool 14 (see FIG. 1) when removing the treatment tool 14.

Furthermore, in the embodiments described above, the fixed valve 45 is connected to the detachable 111 by the connecting band 114 having a rectangular cross-sectional shape. However, the connecting band 114 may be a string-like body having a circular cross-sectional shape or an elliptical cross-sectional shape, for example.

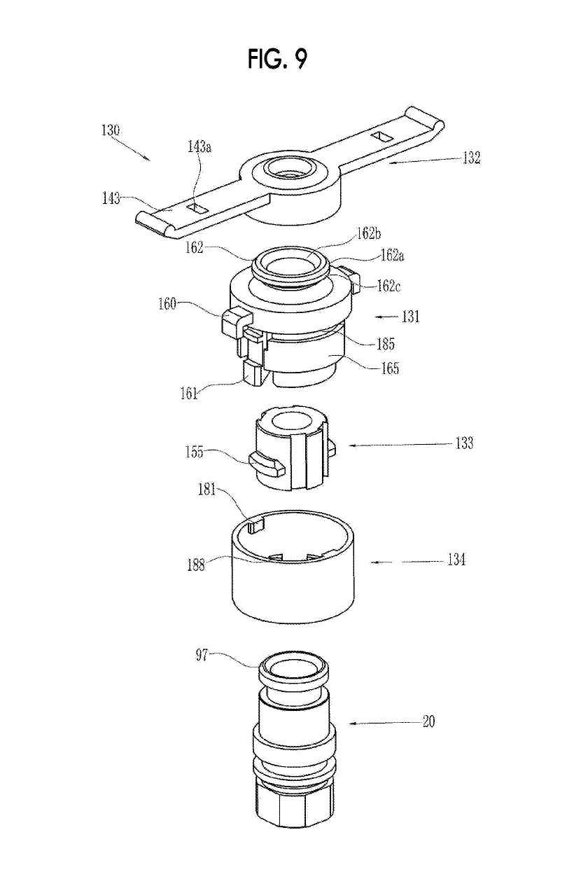

Still further, in the second embodiment, one knob portion 112 is provided in the attachable and detachable valve 111 on the opposite side to the connection band 114, and the engaging claw 113 is provided in the frame body 110 at a position corresponding to the knob portion 112. However, although being omitted in the drawings, a pair of knob portions 143 as shown in FIG. 9 may be provided in a diametrical direction of the attachable and detachable valve 111 perpendicular to the connecting band 114. In this case, a pair of engaging claws 113 shown in FIG. 9 is provided on the outer circumferential surface of the second flange of the frame body.

Next, another example to securely mount the fixed valve and the attachable and detachable valve on the frame body is described with reference to FIGS. 9 to 12. Upon comparison with the embodiments described above, this example is different in that a part of the inner circumferential surface of the frame body faces an insertion path of the treatment tool, and therefore, a material thereof is limited to one having compatibility with the living body.

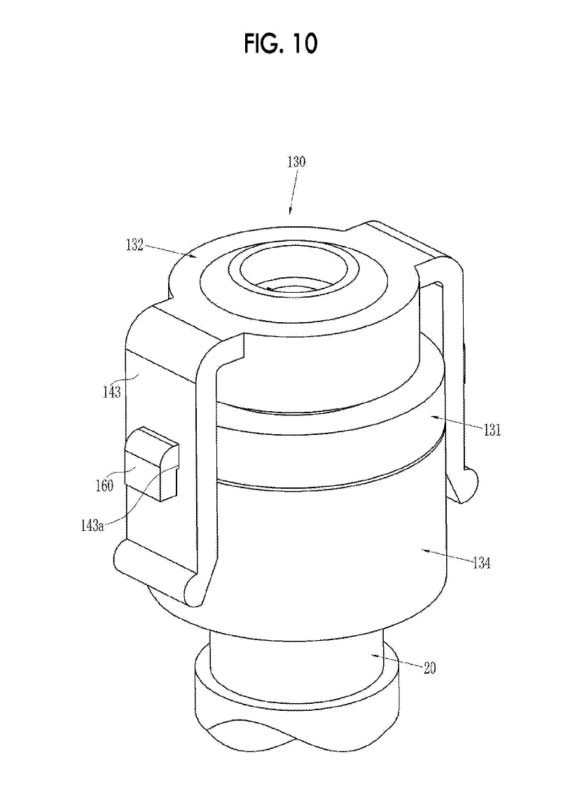

A plug 130 shown in FIGS. 9 and 10 includes a frame body 131, an attachable and detachable valve 132 held by the frame body 131, a fixed valve 133 and a rotation ring 134. The frame body 131 is provided between the attachable and detachable valve 132 and the fixed valve 133.

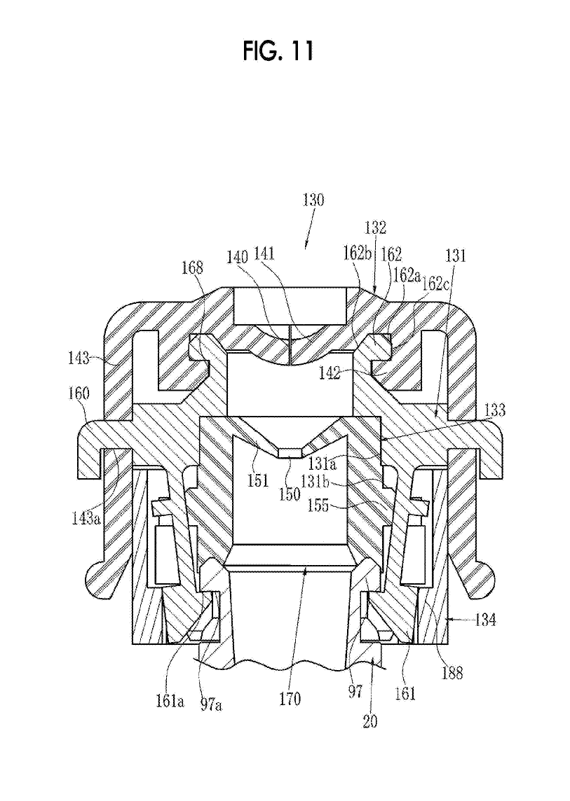

The attachable and detachable valve 132 and the fixed valve 133 are made of elastomer such as rubber. As shown in FIG. 11, the attachable and detachable valve 132 includes a valve film 141 having a slit 140. An annular protrusion 142 protruding inwardly is formed on an inner circumferential surface of the attachable and detachable valve 132. In addition, on an outer circumferential surface of the attachable and detachable valve 132, a pair of knob portions 143 extending in a diametrical direction of the attachable and detachable valve 132 are integrally formed with the attachable and detachable valve 132.

The fixed valve 133 formed in a cylindrical shape is provided with a valve film 151 having a treatment tool insertion hole 150. The valve film 151 is recessed in a hemispherical shape or a conical surface shape, and has the treatment tool insertion hole 150 at a central portion thereof. The fixed valve 133 is inserted into the frame body 131 and held by an inner circumferential surface 131a thereof In addition, an engaging portion 155 which is fixed to and engaged with an engaging groove 131b of the frame body 131 is provided on the outer circumferential surface of the fixed valve 133 in order to prevent the fixed valve 133 from uncoupling.

As shown in FIG. 9, The frame body 131 is integrally made of plastic and includes a pair of engaging claws 160 formed in a direction different from a direction where the treatment tool 14 (see FIG. 1) is pulled out, a fixed claw 161 to be engaged with the mouthpiece 20, a flange 162 and a mounting portion 165 to which the rotation ring 134 is mounted. The flange 162 has a flange outer-side inclined surface 162a on an outer circumferential surface thereof. In addition, a flange inner-side inclined surface 162b is formed on an inner circumferential surface thereof. As shown in FIG. 11, when the flange 162 is covered with the attachable and detachable valve 132, the annular protrusion 142 of the attachable and detachable valve 132 is extendedly deformed so as to get over the flange outer-side inclined surface 162a and the outer circumferential surface 162c, and then being fit into a stepped portion 168.

In the frame body 131, the fixed valve 133 is held by the inner circumferential surface 131a in a state where the fixed valve 133 is in contact with an opening 170 of the mouthpiece 20. The pair of engaging claws 160 which is inserted into engaging holes 143a of the knob portion 143 of the attachable and detachable valve 132 and engaged with the knob portion 143 is provided on the outer circumferential surface of the frame body 131. The fixed claw 161 is formed on a lower end portion of the frame body 131. When the frame body 131 is mounted on the mouthpiece 20, an engaging surface 161a of the fixed claw 161 is engaged with a flange engaging surface 97a.

As shown in FIG. 9, the mounting portion 165 to which the rotation ring 134 is mounted is provided in the frame body 131. Also, a sliding portion 181 provided in the rotation ring 134 slides along a groove portion 185 of the frame body 131, whereby the rotation ring 134 rotates. As shown in FIG. 11, a pressing portion 188 which presses the fixed claw 161 so as to engage the engaging surface 161a of the fixed claw 161 with the flange engaging surface 97a when mounting the frame body 131 on the mouthpiece 20 is provided in the rotation ring 134.

At the time of using the endoscope 10, the plug 130 is mounted on the mouthpiece 20 of the operation portion 12 of the endoscope 10, as shown in FIG. 1. As shown in FIG. 11, this mounting process is carried out by making the fixed valve 133 of the plug 130 be in contact with the opening 170 of the mouthpiece 20, rotating the rotation ring 134 such that the pressing portion 188 of the rotation ring 134 is located at a position of the fixed claw 161, and making the pressing portion 188 press the fixed claw 161. Thereby, the engaging surface 161a of the fixed claw 161 is engaged with the flange engaging surface 97a. The engaging surface 161a is reliably engaged with the flange engaging surface 97a by making the pressing portion 188 press the fixed claw 161, whereby it is not possible to remove the frame body 131 from the mouthpiece 20 in this state.

In a state where the attachable and detachable valve 132 is mounted on the frame body 131 and the treatment tool 14 (see FIG. 1) is not used, the slits 140 of the attachable and detachable valve 132 are in close contact with each other by a resilient force of the attachable and detachable valve 132, and therefore, air- and water-tightness thereof are secured.

When one of the treatment tools 14 having a relatively small diameter is used, it is possible to insert the treatment tool 14 through the slit 140 of the attachable and detachable valve 132 in a state of fitting the attachable and detachable valve 132 into the frame body 131. When the treatment tool 14 is inserted through the slit 140, surface of the slit 140 is in close contact with the outer circumferential surface of the treatment tool 14 by a resilient force of the attachable and detachable valve 132. Therefore, even if the insertion portion 11 is inserted into the body of a patient, blood, body fluid or the like is prevented from leaking outside through the plug 130. Furthermore, when the treatment tool 14 having a large diameter is used, it is also possible to insert the treatment tool 14 through the treatment tool insertion hole 150 of the fixed valve 133 by removing the attachable and detachable valve 132 from the frame body 131.

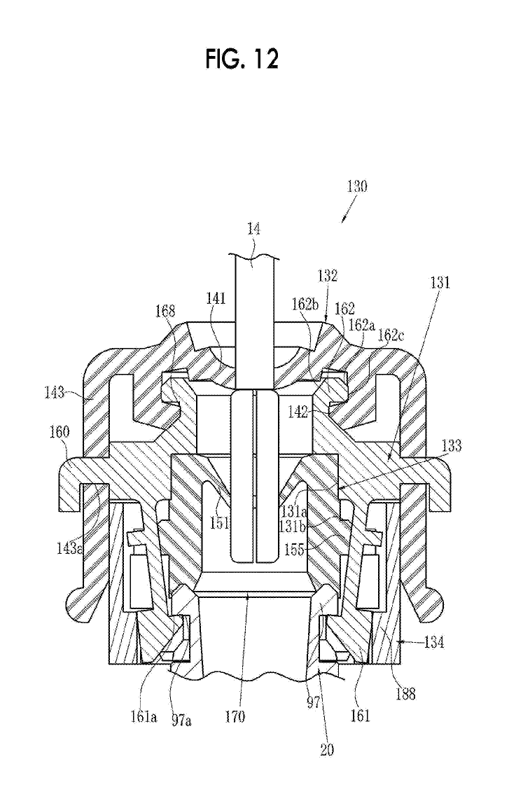

As shown in FIG. 12, since the knob portion 143 is engaged with the engaging claw 160 when pulling out the treatment tool 14 (see FIG. 1), the attachable and detachable valve 132 is prevented from opening. Furthermore, since the knob portion 143 is deformed, the annular protrusion 142 is pushed to the stepped portion 168 side. Thereby, the annular protrusion 142 is further securely in close contact with the stepped portion 168.

The plug 130 is uncoupled from the mouthpiece 20 after the endoscope 10 is used, and then the endoscope 10 is cleaned. When the plug 130 is removed from the mouthpiece 20, the rotation ring 134 is rotated such that the pressing portion 188 of the rotation ring 134 is not located at a position of the fixed claw 161, whereby the engagement between the engaging surface 161a of the fixed claw 161 and the flange engaging surface 97a due to a pressing force by the pressing portion 188 is released. Therefore, it is not possible to rotate the rotation ring 134 after the engagement is released, whereby it is not possible to mount the plug 130 to the mouthpiece 20 again. As a result, reuse thereof is prevented.

According to the plug 130 described above, since the knob portion 143 is engaged with the engaging claw 160, there is no possibility that the attachable and detachable valve 132 may be uncoupled together with the treatment tool 14 by frictional resistance or hooking when removing the treatment tool 14. In addition, since the knob portion 143 is deformed, the annular protrusion 142 is pushed to the stepped portion 168 side. Thereby, the annular protrusion 142 is further securely in close contact with the stepped portion 168.

### Appendix 1

The gist of the plug 130 described above is as follows.

The plug that is mounted on the channel opening portion of the treatment tool channel inside the endoscope and allows the insertion of the treatment tool in the channel opening portion, includes: the frame body that has the engaging portion which is to be engaged with the channel opening portion and the pair of engaging claws which are formed on the outer circumferential surface and protrude in the opposite direction to the pulling-out direction of the treatment tool; the fixed valve made of elastomer having a cylindrical shape that is mounted in the frame body and includes the valve film having the insertion hole for the treatment tool and closes the channel opening portion such that the lower end thereof is in contact with the channel opening portion; and the attachable and detachable valve having a cap shape that includes the valve film having the slit allowing the insertion of the treatment tool, communicates with the fixed valve, and has the pair of knob portions which are formed on the outer circumferential surface and are to be engaged with the engaging claws of the frame body, the engaging holes which are formed on the knob portions at positions where the engaging claws are to be engaged in a state where the knob portions are set to be along the outer circumferential surface of the frame body, and the annular protrusion which is formed on the inner circumferential surface and is externally fit to the stepped portion formed on the opening opposite side to the channel opening portion of the frame body, in which the annular protrusion is pushed to the stepped portion side due to the deformation of the knob portion when pulling out the treatment tool.

What is claimed is:

1. A plug that is mounted on an channel opening portion of a treatment tool channel inside an endoscope and allows an insertion of a treatment tool in the channel opening portion, comprising:
a fixed valve made of elastomer having a cylindrical shape that includes a valve film having an insertion hole for the treatment tool, an engaging portion on an outer circumferential surface and an attachable and detachable valve mounting flange on an upper portion, and is provided in the channel opening portion such that a lower end thereof is in contact with the channel opening portion;
an attachable and detachable valve made of elastomer having a cap shape that includes a valve film having a slit allowing the insertion of the treatment tool and an annular protrusion on an inner circumferential surface to be engaged with the attachable and detachable valve mounting flange, and is attachably and detachably connected to an upper portion of the fixed valve by engaging the annular protrusion with the attachable and detachable valve mounting flange; and
a frame body made of plastic having a cylindrical shape that includes an engaging claw to be engaged with the channel opening portion in a state where a lower end of the fixed valve inserted is in contact with the channel opening portion and a fixed valve mounting flange to be engaged with the engaging portion, and holds the fixed valve by engaging the engaging portion with the fixed valve mounting flange.

2. The plug according to claim 1, further comprising, a connecting portion made of elastomer that is integrally formed with the fixed valve and the attachable and detachable valve so as to combine the fixed valve with the attachable and detachable valve.

3. The plug according to claim 2, further comprising:
a knob portion that is formed on an outer circumferential surface of the attachable and detachable valve and extends in a diametrical direction of the attachable and detachable valve on an opposite side to the connecting portion;
an engaging claw that is formed on the outer circumferential surface of the frame body on an opposite side of the connecting portion in a state where the fixed valve is mounted on the frame body, and extends in a diametrical direction of the frame body as well as protruding in an opposite direction to a pulling-out direction of the treatment tool; and
an engaging hole that is formed on the knob portion at a position where the engaging claw is to be engaged in a state where the knob portion is set to be along the outer circumferential surface of the frame body,
wherein, in a state where the engaging hole of the knob portion is engaged with the engaging claw, a portion corresponding to the length from the attachable and detachable valve of the knob portion to the engaging hole constitutes the connecting portion.

4. The plug according to claim 2, further comprising:
a pair of engaging claws that are formed on the outer circumferential surface of the frame body and extend in a diametrical direction of the frame body as well as protruding in an opposite direction to a pulling-out direction of the treatment tool;
a pair of knob portions that are formed on an outer circumferential surface of the attachable and detachable valve and extend in a diametrical direction of the attachable and detachable valve; and
engaging holes that are formed on the knob portions at positions where the engaging claws are to be engaged in a state where the knob portions are set to be along the outer circumferential surface of the frame body.

5. The plug according to claim 1, further comprising:
a slip-out prevention receiving portion that is integrally formed with the frame body on an outer circumferential surface of the frame body at a lower side of the fixed valve mounting flange of the frame body, and partially covers an outer circumferential surface of the engaging portion.

6. The plug according to claim 2, further comprising:
a slip-out prevention receiving portion that is integrally formed with the frame body on an outer circumferential surface of the frame body at a lower side of the fixed valve mounting flange of the frame body, and partially covers an outer circumferential surface of the engaging portion.

7. The plug according to claim 3, further comprising:
a slip-out prevention receiving portion that is integrally formed with the frame body on an outer circumferential surface of the frame body at a lower side of the fixed valve mounting flange of the frame body, and partially covers an outer circumferential surface of the engaging portion.

8. The plug according to claim 4, further comprising:
a slip-out prevention receiving portion that is integrally formed with the frame body on an outer circumferential surface of the frame body at a lower side of the fixed valve mounting flange of the frame body, and partially covers an outer circumferential surface of the engaging portion.

9. The plug according to claim 1,
wherein the frame body is unable to be separated from the fixed valve by bonding, welding or swaging of partially deforming the frame body.

10. The plug according to claim 2,
wherein the frame body is unable to be separated from the fixed valve by bonding, welding or swaging of partially deforming the frame body.

11. The plug according to claim 3,
wherein the frame body is unable to be separated from the fixed valve by bonding, welding or swaging of partially deforming the frame body.

12. The plug according to claim 4,
wherein the frame body is unable to be separated from the fixed valve by bonding, welding or swaging of partially deforming the frame body.

13. The plug according to claim 5,
wherein the frame body is unable to be separated from the fixed valve by bonding, welding or swaging of partially deforming the frame body.

14. The plug according to claim 1,
wherein the frame body includes knob portions to be separated that protrude in a diametrical direction from an outer circumferential surface on the channel opening portion side, and are connected to the frame body via connection parts positioned at both sides in a circumferential direction,
a notch that is formed on at least one of the connection parts in a direction of a cylindrical center of the frame body, and
a fragile portion that is constituted of the connection part notched, and used for separating the knob portion to be separated.

15. The plug according to claim 2,
wherein the frame body includes knob portions to be separated that protrude in a diametrical direction from an outer circumferential surface on the channel opening portion side, and are connected to the frame body via connection parts positioned at both sides in a circumferential direction,
a notch that is formed on at least one of the connection parts in a direction of a cylindrical center of the frame body, and
a fragile portion that is constituted of the connection part notched, and used for separating the knob portion to be separated.

16. The plug according to claim 3,
wherein the frame body includes knob portions to be separated that protrude in a diametrical direction from an outer circumferential surface on the channel opening portion side, and are connected to the frame body via connection parts positioned at both sides in a circumferential direction,
a notch that is formed on at least one of the connection parts in a direction of a cylindrical center of the frame body, and
a fragile portion that is constituted of the connection part notched, and used for separating the knob portion to be separated.

17. The plug according to claim 4,
wherein the frame body includes knob portions to be separated that protrude in a diametrical direction from an outer circumferential surface on the channel opening portion side, and are connected to the frame body via connection parts positioned at both sides in a circumferential direction,
a notch that is formed on at least one of the connection parts in a direction of a cylindrical center of the frame body, and
a fragile portion that is constituted of the connection part notched, and used for separating the knob portion to be separated.

18. The plug according to claim 5,
wherein the frame body includes knob portions to be separated that protrude in a diametrical direction from an outer circumferential surface on the channel opening portion side, and are connected to the frame body via connection parts positioned at both sides in a circumferential direction,
a notch that is formed on at least one of the connection parts in a direction of a cylindrical center of the frame body, and
a fragile portion that is constituted of the connection part notched, and used for separating the knob portion to be separated.

19. The plug according to claim 14,
wherein the frame body has a side opening that is in contact with the fragile portion and formed in the frame body.

20. The plug according to claim 14,
wherein two fixed claws formed at intervals of approximately 180 degrees in the circumferential direction of the frame body constitute the engaging claw portions, and
wherein the fixed claws are formed at positions symmetrical with respect to a center line of the knob portion to be separated in the diametrical direction.
